(19) United States
(12) Patent Application Publication
(10) Pub. No.: US 2009/0005544 A1
(43) Pub. Date: Jan. 1, 2009

(54) Process for Making Soy Protein Isolates

(76) Inventors: Louis I. Ndife, Columbus, OH (US); Chad R. Cole, Blacklick, OH (US); Jeffrey H. Baxter, Westerville, OH (US)

Correspondence Address: ROSS PRODUCTS DIVISION OF ABBOTT LABORATORIES;DEPARTMENT 108140-DS/1, 625 CLEVELAND AVENUE, COLUMBUS, OH, 43215-1724, US

(21) Appl. No.: 12/145,776

(22) Filed: Jun. 25, 2008

Related U.S. Application Data
(60) Provisional application No. 60/947,142, filed on Jun. 29, 2007.

(51) Int. Cl. C07K 1/30

(52) U.S. Cl. 530/418

(57) ABSTRACT

Disclosed is a process comprising: mixing a soy protein-containing material with an aqueous medium; removing the non-solubilized solids from the slurry to form an alkaline liquid containing solubilized soy protein; adjusting the pH of the solubilized soy protein liquid to between about 3.8 and about 6.6, to precipitate soy protein from the liquid; separating the precipitated soy protein from the pH-adjusted soy protein liquid; resolubilizing the separated soy protein precipitate in an aqueous medium at a pH of from about 1.8 to 3.2; treating the resolubilized soy protein with at least one water-soluble calcium salt to form a calcium-enriched soy protein mixture; precipitating soy protein from the calcium-enriched soy protein mixture by adjusting the pH of the mixture to between about 3.8 and about 6.6; separating the precipitated soy protein from the calcium-enriched soy protein mixture; and washing to form calcium-enriched soy protein isolate having selectively reduced manganese concentrations.

This application claims the benefit of U.S. Provisional Application No. 60/947,142 filed Jun. 29, 2007.

### TECHNICAL FIELD

This invention is directed to a process for making calcium-enriched soy protein isolate comprising selectively reduced manganese concentrations.

### BACKGROUND OF THE INVENTION

Soy proteins have become an increasingly popular source of protein used to fortify the many different types of food products available to consumers today. Such products include salad dressings, soups, imitation meats, beverage powders, cheeses, non-dairy creamer, frozen desserts, whipped topping, breads, breakfast cereals, pastas, infant formulas, and so forth. To achieve soy protein fortification, these products are often formulated with protein-rich soy extracts such as soy protein concentrate, and or soy protein isolates.

Soy protein isolates have become particularly useful in those food products where a highly purified protein source would be most helpful, for example in medical foods, infant formulas, and other nutritional supplements. Soy-based infant formulas, for example, are well known and readily available from a number of commercial sources, including Similac® Isomil® Advance® infant formulas available from Ross Products Division, Abbott Laboratories, Columbus, Ohio. These soy-based formulas utilize soy protein isolate as the sole source of protein.

As a highly purified protein source, soy protein isolates contain at least about 88% protein on a dry weight basis, which is much higher than either soy protein concentrates or soy flours. Soy protein isolates, however, also contain a variety of minerals including calcium, magnesium, iron, copper, and manganese. These inherent mineral concentrations must be accounted for when formulating highly regulated product forms such as infant formulas. It is often a challenge when formulating such products to achieve the targeted mineral concentration, especially when such formulation makes it desirable to use lower concentrations or amounts of any one or more of the minerals inherently found in these soy protein isolates.

### SUMMARY OF THE INVENTION

One embodiment of the invention is directed to a process for making calcium-enriched, soy protein isolates with selectively reduced manganese concentrations. The process comprises: (a) mixing a soy protein-containing material with an aqueous medium, at an alkaline pH, to form a slurry comprising solubilized soy protein and non-solubilized solids; (b) removing the non-solubilized solids from the slurry to form an alkaline liquid containing solubilized soy protein; (c) adjusting the pH of the solubilized soy protein liquid to between about 3.8 and about 6.6, to precipitate soy protein from the liquid; (d) separating the precipitated soy protein from the pH-adjusted soy protein liquid; (e) resolubilizing the separated soy protein precipitate in an aqueous medium at a pH of from about 1.8 to 3.2; (f) treating the resolubilized soy protein with at least one water-soluble calcium salt to form a calcium-enriched soy protein mixture; (g) precipitating soy protein from the calcium-enriched soy protein mixture by adjusting the pH of the mixture to between about 3.8 and about 6.6; (h) separating the precipitated soy protein from the calcium-enriched soy protein mixture; and then (i) washing the precipitated soy protein to form a calcium-enriched soy protein isolate having selectively reduced manganese concentrations.

It has now been discovered that the process as described herein results in the manufacture of a soy protein isolate having a calcium-enriched matrix that, paradoxically, has selectively reduced manganese content is not completely understood why manganese concentrations are selectively reduced by the process while other less reactive minerals, such as copper, iron and zinc, are either not reduced at all or to a much less extent.

The calcium-enriched soy protein isolates made in accordance with the described processes are especially useful in infant formulas or other nutritional products in which selective reduction of manganese content would be desirable.

### DETAILED DESCRIPTION OF THE INVENTION

The various embodiments of the present invention comprise a series of essential process steps, each of which is described in detail hereinafter.

The term “soy protein isolate” as used herein, unless otherwise specified, refers to soy bean extracts comprising at least about 88%, typically between about 90% and 95%, soy protein on a dry weight basis.

The term “soy protein-containing material” as used herein, unless otherwise specified, includes any material comprising soy beans or soy bean extract that are suitable for manufacturing soy protein isolates, non-limiting examples of which is defatted soy flour, defatted soy flakes, defatted soy nuts, defatted soy meal, defatted soy grits, and combinations thereof.

All percentages, parts and ratios as used herein, are by weight of the total composition, unless otherwise specified. All such weights as they pertain to listed ingredients are based on the active level and, therefore, do not include solvents or by-products that may be included in commercially available materials, unless otherwise specified.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All references to singular characteristics or limitations of the present invention shall include the corresponding plural characteristic or limitation, and vice versa, unless otherwise specified or dearly implied to the contrary by the context in which the reference is made.

All combinations of method or process steps as used herein may be performed in any order, unless otherwise specified or clearly implied to the contrary by the context in which the referenced combination is made.

The various embodiments described herein may be substantially free of any optional or selected essential ingredient or feature described herein, provided that the remaining process still contains all of the required features as described herein. In this context, and unless otherwise specified, the term “substantially free” means that the selected process contains less than a functional amount or degree of a selected ingredient or feature. From a composition standpoint, “substantially free” means that the recited compositional feature may contain less than 0.1% by weight, and also including zero percent by weight, of such optional or selected compositional feature.

The process embodiments described herein may comprise, consist of, or consist essentially of the essential elements of the embodiments described herein, as well as any additional or optional ingredients, components, or limitations described herein or otherwise useful for soy protein isolate process applications.

### Process

The process embodiments of the present invention provide a method for making calcium-enriched, soy protein isolate comprising selectively reduced levels of manganese.

A first step of the process comprises mixing a soy protein-containing material with an aqueous medium, at an alkaline pH, to form an alkaline slurry comprising solubilized soy protein and non-solubilized solids.

During this first process step, the alkaline slurry may initially comprise up to about 30%, including from about 5% to about 15%, by weight of the soy protein-containing material.

Also during this first process step, the aqueous medium may be adjusted to have an alkaline pH of at least about 7.0, including from about 7.5 to about 9.0, and also including from about 8.0 to about 9.0. The aqueous medium may be adjusted to have the requisite pH prior, during, or after combining the medium and the soy-protein containing material.

Also, during the first process step, the alkaline slurry may be stirred or agitated for a period of time, and at a temperature, at least sufficient to form the above-described soluble and insoluble fractions. For example, the slurry may be stirred for up to about 60 minutes, more typically from about 30 minutes to about 40 minutes, at a slurry temperature of up to about 150° F., typically between about 80° F. and about 150° F., more typically between about 80° F. to about 100° F.

Suitable soy protein-containing materials for use in the first process step include any soy-protein source known for or otherwise suitable for use in the manufacture of a soy protein isolate. The soy protein-containing material may be defatted and desolventized, non-limiting examples of which include defatted soy flour, defatted soy flakes, defatted soy nuts, defatted soy meal, defatted soy grits, or combinations thereof. Non-limiting examples of some suitable sources of soy protein-containing starting materials include defatted soy flakes from Cargill, Inc., Minneapolis, Minn., USA; and defatted soy flakes from Archer Daniels Midland Company, Decatur, Ill., USA.

A second step of the process comprises removing non-soluble solids from the resulting alkaline slurry of the first process step to form an alkaline liquid containing solubilized soy protein and typically at least some minerals. The insoluble solids thus removed, which may be discarded or used for other purposes, typically include undissolved solids such as spent soy flakes, spent soy flour, spent soy grits, insoluble protein, insoluble fiber, or combinations thereof.

The second process step may result in the removal of from about 50% to 100%, including from about 75% to 98%, and also including from about 85% to about 97%, by weight of total non-solubilized solids in the alkaline liquid. The non-solubilized solids may be removed by any one of a number of physical separation techniques, non-limiting examples of which include centrifugation, filtration, or combinations thereof. Centrifugation is preferred.

The slurry may be centrifuged, for example, at between about 2,000 and 20,000 revolutions per minute (rpm), most typically between about 4,000 rpm and 7,000 rpm, for a period of time sufficient to effect the desired separation, most typically within about 20 minutes, more typically within about 7 minutes to about 16 minutes.

A third step of the process comprises the precipitation of soy protein from the resulting soy protein liquid (from the second process step). This may be accomplished by adjusting or reducing the pH of the solubilized soy protein liquid to the extent necessary to realize protein precipitation, which is most typically between about 3.8 and about 6.6, more typically between about 4.0 and about 5.0, most typically at or below about 4.5 or the approximate isoelectric point of the solubilized soy protein.

A fourth step of the process comprises the separation of the precipitated soy protein from the pH-adjusted soy protein liquid (from the third process step). The precipitated soy protein may be separated from the pH-adjusted liquid by any one of a number of physical separation techniques, non-limiting examples of which include centrifugation, filtration, or combinations thereof. Centrifugation is preferred. The precipitated soy protein thus removed is in the form of moist curds.

A fifth step of the process comprises resolubilizing the separated soy protein precipitate or curd (from the fourth process step) in an aqueous medium at a pH sufficiently low to resolubilize typically at least 80%, more typically from about 80% to about 99%, and even more typically from about 90% to about 99%, by weight of the protein into solution. To accomplish such resolubilization, the pH is typically adjusted to between about 1.8 and 3.2, more typically from about 2.0 to 3.0, and even more typically from about 2.2 to 2.7. These specified pH ranges tend to allow for the solubilization of a significant amount of soy protein, which facilitates the displacement of manganese during the subsequent calcium treatment step.

During the fifth process step, the precipitated soy protein may be resolubilized in the aqueous medium, most typically distilled or other suitable form of water, to yield a weight ratio of soy protein to water of from about 1:10 to about 10:1, including of from about 1:3 to about 1:1.

A sixth step of the process comprises the treatment of the resolubilized soy protein (from the fifth process step) with at least one water-soluble calcium source to form a calcium-enriched soy protein mixture. The water-soluble calcium source may include one or more of calcium chloride, calcium lactate, calcium gluconate, or other calcium-containing material or salt that is water-soluble or is otherwise rendered water soluble within the calcium-enriched soy protein mixture. Calcium chloride is preferred. Non-limiting examples of some suitable sources of water-soluble calcium salts include calcium chloride from Degussa Food Ingredients, Champagne, Ill., USA; and calcium chloride from AerChem Inc., Bloomington, Ind., USA.

In this context, the term “water soluble” means that the calcium source is at least as water soluble as one or more of calcium chloride, calcium lactate, or calcium gluconate.

Also during the sixth process step, the resolubilized soy protein of step five may be treated with at least one water-soluble calcium salt in an amount ranging from about 0.0001 g to about 0.1 g, including from about 0.0005 g to about 0.05 g, and also including from about 0.001 g to about 0.01 g, of calcium per gram of protein in the mixture. The specified amount of calcium per gram of protein is calculated based on the calcium content in the calcium containing salt.

Higher calcium treatment levels may, however, allow for shorter reaction times and lower temperatures, and vice versa. In this context, the resolubilized soy protein of step five may be treated with at least one water-soluble calcium salt for a period of time of at least about 3 minutes, including from about 3 to about 20 minutes, and also including from about 5 to about 10 minutes. In a preferred embodiment of the present invention, the temperature of the resolubilized soy protein, prior to treatment with at least one water-soluble calcium salt, is adjusted to from about 70 to about 130° F., preferably from about 80 to about 100° F.

A seventh step of the process comprises the precipitation of soy protein from the calcium-enriched soy protein mixture (from the sixth process step). This may be accomplished by adjusting or reducing the pH of the solubilized soy protein liquid to the extent necessary to realize protein precipitation, which is most typically between about 3.8 and about 6.6, more typically between about 4.0 and about 5.0, most typically at or below about 4.5 or the approximate isoelectric point of the solubilized soy protein.

An eighth step of the process comprises separating the precipitated soy protein from the calcium-enriched soy protein mixture. The precipitated soy protein may be separated from the calcium-enriched soy protein mixture by any one of a number of physical separation techniques, non-limiting examples of which include centrifugation, filtration, or combinations thereof. Centrifugation is preferred.

A ninth step of the process comprises washing the precipitated soy protein of step eight to form a soy protein isolate. Any known or otherwise suitable technique for washing soy protein precipitates may be used herein, for example by resuspending the precipitated soy protein in distilled water to yield a weight ratio of soy protein to water of from about 1:10 to about 10:1, including from about 1:3 to about 1:1, and subsequently centrifuging to recover the resulting soy protein isolate.

In a preferred embodiment the resulting soy protein isolate may be neutralized, after washing, by adjusting the pH as necessary, typically between about 5.5 and about 8.5, more typically from about 6.0 and about 8.0, and even more typically from about 6.5 and about 7.5.

In yet another preferred embodiment, the soy protein isolate resulting from the processes herein may be dried by any known or otherwise effective technique for forming soy protein isolate powders. To accomplish the desired drying, the soy protein isolate, may be resuspended in an aqueous medium and subsequently subjected to a suitable drying step, non-limiting examples of which include belt-drying, freeze-drying, drum-drying, spray-drying, or combinations thereof. Spray drying is preferred. The soy protein isolate is preferably neutralized prior to drying.

In another preferred embodiment, prior to drying the soy protein isolate of step nine, the soy protein isolate is pasteurized at a temperature of, for example, between about 80° C. and about 135° C.

The various solutions used in the process described herein may further comprise other additional ingredients suitable for use as manufacturing aids, or which are otherwise known or effective for use in the manufacture of soy protein isolates, provided that such other additional ingredients are compatible with the ingredients of the corresponding solution, or which do not unduly impair the process embodiments of the present invention and the intended benefits arising therefrom.

Non-limiting examples of optional ingredients for use in the process described herein include pH buffering agents (especially potassium hydroxide and hydrochloric add), processing aides, washing agents, masking agents, modifying agents, and so forth. Food grade reagents, agents, and ingredients are most typically used.

### Calcium-Enriched Soy Protein Isolate

The process described herein provides a method for making soy protein isolates that are calcium-enriched and contain selectively reduced concentrations or amounts of manganese.

In this context, “calcium-enriched” means that the soy protein isolate has a higher calcium concentration compared to that of the soy protein-containing material from which it was derived. The calcium-enriched soy protein isolates described herein may have a calcium concentration that is at least about 20% more by weight of protein, on a dry weight basis, than that of the corresponding soy protein-containing material, more typically from about 20% to about 500%, including from about 45% to about 400%, also including from about 80% to about 320%, and also including from about 100% to about 200%, by weight of protein, on a dry weight basis.

The soy protein isolate made in accordance with the method herein may also have a selectively reduced manganese concentration compared to that of the soy protein-containing material from which it was derived. The soy protein isolate described herein may have a manganese concentration that is reduced by at least about 20% by weight of protein, on a dry weight basis, of the manganese content or concentration of the corresponding soy protein-containing material, typically from about 20% to about 99%, more typically from about 60% to about 98%, and even more typically from about 70% to about 98%, by weight of protein, on a dry weight basis.

The soy protein isolate may also have the above-manganese reductions without subsequent reductions in one or more, most typically all, of zinc, iron, and copper concentrations or content, or if there are reductions in any one of zinc, iron, or copper in the soy protein isolate, such reductions are to a lesser magnitude than that of manganese.

The following Studies further describe and demonstrate specific, but non-limiting, embodiments within the scope of the present invention. Many variations thereof are possible without departing from the spirit and scope of the invention.

### Study 1

A study is conducted to evaluate the removal (displacement) of manganese, by the addition of different levels of calcium chloride (CaCl₂), during manufacture of soy protein isolate. The following three levels of calcium chloride are tested: 1) 0 v/v % of 1M calcium chloride; 2) 10 v/v % of 1M calcium chloride; and 3) 20 v/v % of 1M calcium chloride.

Defatted and desolventized white soy flakes are obtained from Cargill, Inc., Minneapolis, Minn., USA. The soy flakes are added to water (80-100° F.) to form a slurry containing 5 w/v % of total solids. The pH of the slurry is adjusted to 8.5 with 1N KOH. The slurry is stirred for a period of 30 minutes. The slurry is centrifuged at 5000 rpm for 10 minutes. The supernatant, which contains solubilized, soy protein, is separated from the pellet. The supernatant is then split into three parts (parts A, B, and C).

### Part A (Sample A=0 v/v % of 1M Calcium Chloride):

The pH of the part A supernatant (slurry) is adjusted to 4.5 with 1N HCL to precipitate soy protein. The slurry is centrifuged at 5000 rpm for 10 minutes. The supernatant is removed and the precipitated soy protein is collected. The precipitated soy protein is washed by resuspending the precipitated soy protein in distilled water to yield a weight ratio of soy protein to water of 1:3 and subsequently centrifuging to recover soy protein. Samples of the soy protein isolate are then analyzed for protein using the Kjeldahl method and for minerals using the inductively couple plasma (ICP) method.

### Part B (Sample B=10 v/v % of 1M Calcium Chloride):

The pH of the part B supernatant (slurry) is adjusted to 2.5 with 1N HCL. 10 v/v % of 1M calcium chloride is added to the slurry. The slurry is then stirred for a period of 5 minutes. The pH is adjusted to 4.5 with 1N KOH to precipitate soy protein. The slurry is centrifuged at 5000 rpm for 10 minutes. The supernatant is removed and the precipitated soy protein is collected. The precipitated soy protein is washed by resuspending the precipitated soy protein in distilled water to yield a weight ratio of soy protein to water of 1:3 and subsequently centrifuging to recover soy protein. Samples of the soy protein isolate are then analyzed for protein using the Kjeldahl method and for minerals using the inductively couple plasma (ICP) method.

### Part C (Sample C=20 v/v % of 1M Calcium Chloride):

The pH of the part C supernatant (slurry) is adjusted to 2.5 with 1N HCL. 20 v/v % of 1M calcium chloride is added to the slurry. The slurry is then stirred for a period of 5 minutes. The pH is adjusted to 4.5 with 1N KOH to precipitate soy protein. The slurry is centrifuged at 5000 rpm for 10 minutes. The supernatant is removed and the precipitated soy protein is collected. The precipitated soy protein is washed by resuspending the precipitated soy protein in distilled water to yield a weight ratio of soy protein to water of 1:3 and subsequently centrifuging to recover soy protein. Samples of the soy protein isolate are then analyzed for protein using the Kjeldahl method and for minerals using the inductively couple plasma (ICP) method.

The protein and minerals results are summarized in the following table:

| | Sample A - | | |
|---|---|---|---|
| | Control | Sample B | Sample C |
| | 0 v/v % 1M | 10 v/v % 1M | 20 v/v % 1M |
| ASSAY | CaCl₂ | CaCl₂ | CaCl₂ |
| Protein (g/100 g dry wt. | 63.5 | 62.2 | 62.1 |
| basis) | | | |
| Ca (mg/g protein) | 0.594 | 3.220 | 4.27 |
| Cu (mg/g protein) | 0.003 | 0.002 | 0.002 |
| Fe (mg/g protein) | 0.037 | 0.038 | 0.037 |
| Mn (mg/g protein) | 0.003 | 0.006 | 0.005 |
| Mg (mg/g protein) | 0.150 | 0.077 | 0.056 |

As shown in the above data table, calcium levels in finished product soy protein isolate increase upon addition of calcium chloride during the manufacture of soy protein isolate. However, unexpectedly, copper (Cu), iron (Fe), and manganese (Mn) levels remain unchanged.

It is well known to one of ordinary skill in the art that metals that are more active displace the ions of less active metals in aqueous solutions. Therefore, Applicants expect that calcium would displace manganese, which would result in decreased levels of manganese in the final soy protein isolate. However, this expected result is not observed.

### Study 2

A second study is conducted to evaluate the impact of increasing molar concentrations of calcium chloride on the displacement of manganese. The following two levels of calcium chloride are tested: 1) 5 v/v % of 1M calcium chloride; and 2) 5 v/v % of 4M calcium chloride.

Defatted and desolventized white soy flakes are obtained from Cargill, Inc., Minneapolis, Minn., USA The soy flakes are added to water (80-100° F.) to form a slurry containing 5 w/v % of total solids. The pH of the slurry is adjusted to 8.5 with 1N KOH. The slurry is stirred for a period of 30 minutes. The slurry is centrifuged at 5000 rpm for 10 minutes. The supernatant, which contains solubilized, soy protein, is separated from the pellet. The supernatant is then split into two parts (parts A and B).

### Part A (Sample A=5 v/v % of 1M Calcium Chloride):

The pH of the part A supernatant (slurry) is adjusted to 2.5 with 1N HCL. 5 v/v % of 1M calcium chloride is added to the slurry. The slurry is then stirred for a period of 5 minutes. The pH is adjusted to 4.5 with 1N KOH to precipitate soy protein. The slurry is centrifuged at 5000 rpm for 10 minutes. The supernatant is removed and the precipitated soy protein is collected. The precipitated soy protein is washed by resuspending the precipitated soy protein in distilled water to yield a weight ratio of soy protein to water of 1:3 and subsequently centrifuging to recover soy protein. Samples of the soy protein isolate are then analyzed for protein using the Kjeldahl method and for minerals using the inductively couple plasma (ICP) method.

### Part B (Sample B=5 v/v % of 4M Calcium Chloride):

The pH of the part B supernatant (slurry) is adjusted to 2.5 with 1N HCL. 5 v/v % of 4M calcium chloride is added to the slurry. The slurry is then stirred for a period of 5 minutes. The pH is adjusted to 4.5 with 1N KOH to precipitate soy protein. The slurry is centrifuged at 5000 rpm for 10 minutes. The supernatant is removed and the precipitated soy protein is collected. The precipitated soy protein is washed by resuspending the precipitated soy protein in distilled water to yield a weight ratio of soy protein to water of 1:3 and subsequently centrifuging to recover soy protein. Samples of the soy protein isolate are then analyzed for protein using the Kjeldahl method and for minerals using the inductively couple plasma (ICP) method.

The protein and minerals results are summarized in the following table:

| | Sample A | Sample B |
|---|---|---|
| ASSAY | 5 v/v % 1M CaCl₂ | 5 v/v % 4M CaCl₂ |
| Protein (g/100 g dry wt. | 54.2 | 54.1 |
| basis) | | |
| Ca (mg/g protein) | 5.5 | 12.4 |
| Cu (mg/g protein) | 0.001 | 0.001 |
| Fe (mg/g protein) | 0.041 | 0.014 |
| Mn (mg/g protein) | 0.005 | 0.004 |
| Zn (mg/g protein) | 0.014 | 0.016 |
| Mg (mg/g protein) | 0.142 | 0.127 |

As shown in the above data table, calcium levels in finished product soy protein isolate increase upon addition of calcium chloride during the manufacture of soy protein isolate. However, copper, iron, zinc, and manganese levels unexpectedly remain relatively unchanged, despite an increase in the molar concentration of the added calcium chloride.

It is well known to one of ordinary skill in the art that metals that are more active displace the ions of less active metals in aqueous solutions. Therefore, Applicants expect that calcium would displace manganese, which would result in decreased levels of manganese in the final soy protein isolate. However, this expected result is not observed.

### Study 3

Conventional processes for the manufacture of soy protein isolate typically comprise only one precipitation step wherein soy protein is precipitated from a solubilized soy protein-containing liquid. According to the methods of Studies 1 and 2, calcium chloride is added, during the manufacture of soy protein isolate, directly to a solubilized soy protein-containing liquid extract prior to the precipitation of protein from the solubilized soy protein-containing liquid.

This Study 3 is now conducted to evaluate whether an initial precipitation step, prior to the addition of calcium chloride, is necessary during the manufacture of soy protein isolate, in order for calcium chloride to displace manganese.

The following three soy protein isolates are manufactured and tested: 1) control soy protein isolate manufactured using a process comprising a single precipitation step and no calcium addition (0 v/v % of 1M calcium chloride); 2) soy protein isolate manufactured using a process comprising two precipitation steps, wherein the addition of 5 v/v % of 1M calcium chloride occurs after an initial precipitation step; and 3) soy protein isolate manufactured using a process comprising no calcium addition (0 v/v % of 1M calcium chloride) and two precipitation steps.

Defatted and desolventized white soy flakes are obtained from Cargill, Inc., Minneapolis, Minn., USA. The soy flakes are added to water (80-100° F.) to form a slurry containing 5 w/v % of total solids. The pH of the slurry is adjusted to 8.5 with 1N KOH. The slurry is stirred for a period of 30 minutes. The slurry is centrifuged at 5000 rpm for 10 minutes. The supernatant, which contains solubilized, soy protein, is separated from the pellet. The supernatant is then split into three parts (parts A, B, and C).

### Part A (Sample A=0 v/v % of 1M Calcium Chloride, One Precipitation Step)

The pH of the part A supernatant (slurry) is adjusted to 4.5 with 1N HCL to precipitate soy protein. The slurry is centrifuged at 5000 rpm for 10 minutes. The supernatant is removed and the precipitated soy protein is collected. The precipitated soy protein is washed by resuspending the precipitated soy protein in distilled water to yield a weight ratio of soy protein to water of 1:3 and subsequently centrifuging to recover soy protein. Samples of the soy protein isolate are then analyzed for protein using the Kjeldahl method and for minerals using the inductively couple plasma (ICP) method.

### Part B (Sample B=5 v/v % of 1M Calcium Chloride, Two Precipitation Steps):

The pH of the part B supernatant (slurry) is adjusted to 4.5 with 1N HCL to precipitate soy protein. The slurry is centrifuged at 5000 rpm for 10 minutes. The supernatant is removed and the precipitated soy protein is collected. The precipitated soy protein is resolubilized in distilled water to yield a weight ratio of soy protein to water of 1:3. The pH of the resultant slurry is then adjusted to 2.5 with 1N HCL. 5 v/v % of 1M calcium chloride is added to the slurry. The slurry is then stirred for a period of 5 minutes. The pH is adjusted to 4.5 with 1N KOH to precipitate soy protein. The slurry is centrifuged at 5000 rpm for 10 minutes. The supernatant is removed and the precipitated soy protein is collected. The precipitated soy protein is washed by resuspending the precipitated soy protein in distilled water to yield a weight ratio of soy protein to water of 1:3 and subsequently centrifuging to recover soy protein. Samples of the soy protein isolate are then analyzed for protein using the Kjeldahl method and for minerals using the inductively couple plasma (ICP) method.

### Part C (Sample C=0 v/v % of 1M Calcium Chloride, Two Precipitation Steps):

The pH of the part C supernatant (slurry) is adjusted to 4.5 with 1N HCL to precipitate soy protein. The slurry is centrifuged at 5000 rpm for 10 minutes. The supernatant is removed and the precipitated soy protein is collected. The precipitated soy protein is resolubilized in distilled water to yield a weight ratio of soy protein to water of 1:3. The pH of the resultant slurry is then adjusted to 2.5 with 1N HCL. The slurry is then stirred for a period of 5 minutes. The pH is adjusted to 4.5 with 1N KOH to precipitate soy protein. The slurry is centrifuged at 5000 rpm for 10 minutes. The supernatant is removed and the precipitated soy protein is collected. The precipitated soy protein is washed by resuspending the precipitated soy protein in distilled water to yield a weight ratio of soy protein to water of 1:3 and subsequently centrifuging to recover soy protein. Samples of the soy protein isolate are then analyzed for protein using the Kjeldahl method and for minerals using the inductively couple plasma (ICP) method.

The protein and minerals results are summarized in the following table:

| | Sample A - | | |
|---|---|---|---|
| | Control | Sample B | Sample C |
| | Single | Double | Double |
| | precipitation | precipitation | precipitation |
| ASSAY | 0 v/v % CaCl₂ | 5 v/v % CaCl₂ | 0 v/v % CaCl₂ |
| Protein (g/100 g dry | 72.4 | 68.5 | 69.9 |
| wt. basis) | | | |
| Ca (mg/g protein) | 0.485 | 1.460 | 0.492 |
| Cu (mg/g protein) | 0.002 | 0.002 | 0.002 |
| Fe (mg/g protein) | 0.019 | 0.027 | 0.022 |
| Mn (mg/g protein) | 0.002 | 0.0006 | 0.002 |
| Zn (mg/g protein) | 0.004 | 0.006 | 0.004 |
| Mg (mg/g protein) | 0.142 | 0.021 | 0.079 |

As shown in the above data table, calcium levels in finished product soy protein isolate increase and manganese levels decrease upon the addition of calcium chloride after an initial precipitation step (Sample B).

Unexpectedly, although manganese levels decrease upon the addition of calcium chloride during the manufacture of soy protein isolate, zinc, iron, and copper levels remain relatively unchanged. It is well known to one of ordinary skill in the art that metals that are more active displace the ions of less active metals in aqueous solutions. According to the Activity Series of the metals, zinc, iron, and copper are all less active than manganese. Therefore, it is surprising that manganese—a more reactive metal than Zn, Fe, and Cu—is selectively displaced by calcium, while less reactive metals (Zn, Fe, and Cu) are not displaced.

### CONCLUSIONS

The above studies show that the addition of at least one water-soluble calcium salt, during the manufacture of soy protein isolate, results in the selective displacement of manganese provided that the water-soluble calcium salt is employed in a double precipitation sequence as described herein. Other minerals that are less reactive than manganese are not displaced by calcium. This result is unexpected since it is generally known that metals that are more active displace the ions of less active metals in aqueous solutions.

Furthermore, the above studies show that the soy protein isolate process embodiments of the present invention, which comprise the addition of at least one water-soluble calcium salt after an initial precipitation step, are unique in allowing for the selective displacement of manganese by calcium. Unexpectedly, the above studies show that the selective displacement of manganese occurs if at least one water-soluble calcium salt is added after a precipitation step. If water-soluble calcium salt is added directly to a solubilized soy protein-containing liquid prior to the precipitation of protein from the solubilized soy protein-containing liquid, manganese will not be displaced.

What is claimed is:

1. A process comprising:
(a) mixing a soy protein-containing material with an aqueous medium, at an alkaline pH, to form a slurry comprising solubilized soy protein and non-solubilized solids;
(b) removing the non-solubilized solids from the slurry to form an alkaline liquid containing solubilized soy protein;
(c) adjusting the pH of the solubilized soy protein liquid to between about 3.8 and about 6.6, to precipitate soy protein from the liquid;
(d) separating the precipitated soy protein from the pH-adjusted soy protein liquid;
(e) resolubilizing the separated soy protein precipitate in an aqueous medium at a pH of from about 1.8 to 3.2;
(f) treating the resolubilized soy protein with at least one water-soluble calcium salt to form a calcium-enriched soy protein mixture;
(g) precipitating soy protein from the calcium-enriched soy protein mixture by adjusting the pH of the mixture to between about 3.8 and about 6.6;
(h) separating the precipitated soy protein from the calcium-enriched soy protein mixture; and then
(i) washing the precipitated soy protein to form a soy protein isolate.

2. The process of claim 1, wherein the slurry of step (a) contains from about 5% to about 30% by weight of the soy protein-containing material.

3. The process of claim 1, wherein the pH of the liquid of step (c) and the pH of the mixture of step (g) are adjusted to between about 4.0 and about 5.0.

4. The process of claim 1, wherein the soy protein-containing material of step (a) comprises defatted soy flakes.

5. The process of claim 1, wherein the water-soluble calcium salt is selected from the group consisting of calcium chloride, calcium lactate, calcium gluconate, and mixtures thereof.

6. The process of claim 1, wherein the water-soluble calcium salt comprises calcium chloride.

7. The process of claim 1 wherein the resolubilized soy protein of step (f) is treated with the water-soluble calcium salt in an amount ranging from about 0.001 to about 0.010 g of calcium per gram of protein in the mixture.

8. The process of claim 1, wherein the resolubilized soy protein of step (f) is treated with the water-soluble calcium salt for a period of time of ranging from about 3 to about 20 minutes.

9. The process of claim 1, wherein the soy protein isolate is neutralized.

10. The process of claim 1, wherein the soy protein isolate is dried to form a soy protein isolate powder.

11. The process of claim 1, wherein the soy protein isolate is neutralized and dried to form a soy protein isolate powder.

12. The process of claim 10, wherein the drying comprises spray-drying.

13. A process comprising:
(a) mixing a soy protein-containing material with an aqueous medium, at a pH of from about 8 to about 9, to form a slurry comprising solubilized soy protein and non-solubilized solids;
(b) removing by centrifugation the non-solubilized solids from the slurry to form an alkaline liquid containing solubilized soy protein;
(c) precipitating soy protein from the centrifuged alkaline liquid by adjusting the pH of the liquid to between about 4 and about 5;
(d) separating by centrifugation the precipitated soy protein from the pH-adjusted soy protein liquid;
(e) resolubilizing the precipitated soy protein in an aqueous medium at a pH of from about 2.0 to 3.0;
(f) treating the resolubilized soy protein with calcium chloride for a period of time of from about 3 and 20 minutes, to form a calcium-enriched soy protein mixture;
(g) precipitating soy protein from the calcium-enriched soy protein mixture by adjusting the pH of the mixture to between about 4 and about 5;
(h) removing by centrifugation the precipitated soy protein from the calcium-enriched soy protein mixture; and then
(i) washing the precipitated soy protein of step (h) to form a soy protein isolate.

14. The process of claim 13 wherein the slurry initially comprises from about 5% to about 30% by weight of the soy-protein containing material.

15. The process of claim 13, wherein the resulting soy protein isolate is subsequently neutralized.

16. The process of claim 13, wherein the resulting soy protein isolate is spray dried to form a soy protein isolate powder.

17. The process of claim 13, wherein the soy protein isolate is neutralized and then spray dried to form a soy protein isolate powder.

18. The process of claim 13 wherein the resolubilized soy protein of step (f) is treated with calcium chloride in an amount ranging from about 0.001 to about 0.010 g of calcium per gram of protein in the mixture.
